(19) United States
(12) Patent Application Publication
(10) Pub. No.: US 2026/0317683 A1
(43) Pub. Date: Oct. 15, 2026

(54) INCUBATOR

(71) Applicant: SHIBUYA CORPORATION, Kanazawa-shi (JP)

(72) Inventors: Kazuhito TANIMOTO, Kanazawa-shi (JP); Naoya DEGUCHI, Kanazawa-shi (JP); Masaru ISHII, Kanazawa-shi (JP); Masaki NAKANO, Kanazawa-shi (JP)

(21) Appl. No.: 19/641,916

(22) Filed: Apr. 8, 2026

(30) Foreign Application Priority Data
Apr. 11, 2025 (JP) 2025-065390

(51) Int. Cl. C12M 1/00

(52) U.S. Cl. CPC C12M 41/14

(57) ABSTRACT

An incubator including housing chambers for housing culture containers therein. The incubator has an opening/closing member, and a breathable wall having a plurality of gas supply ports is provided on rear faces of the housing chambers. The incubator includes a gas pressurizing chamber at a rear of the housing chambers and communicating therewith via the gas supply ports, a gas merging chamber in communication with the gas pressurizing chamber, and a gas delivery unit for purifying gas in the gas merging chamber and delivering the purified gas to the gas pressurizing chamber. An exhaust passage allows individual communication between each housing chamber and the gas merging chamber, so that gas in each housing chamber is discharged into the gas merging chamber via the exhaust passage.

### BACKGROUND OF THE INVENTION

### Field of the Invention

The present invention is related to an incubator and, more specifically, to an incubator including a plurality of housing chambers for housing culture containers therein.

### Description of the Related Art

It is a known practice to culture cells by housing a culture container housing cells therein in an incubator, with the interior of the incubator maintained in an environment suitable for culturing. In recent years, for the purpose of culturing a large amount of cells, an incubator including a plurality of housing chambers capable of housing culture containers therein is known (Japanese Laid-Open Patent Application No. 2022-184127).

This incubator makes it possible to sequentially house, into the housing chambers, the culture containers in which cells are seeded and to sequentially take the culture containers out of the housing chambers in the order in which they have reached their culture periods. In doing so, as each of the housing chambers can be individually opened and closed with an opening/closing member, it is possible to house and to take out certain culture containers, without affecting the other culture containers being cultured.

In addition, in the incubator disclosed in Japanese Laid-Open Patent Application No. 2022-184127, gas supply ports are provided in the rear faces of the housing chambers, and a gas pressurizing chamber is provided at the rear of the housing chambers so as to communicate with the housing chambers via the gas supply ports. Thus, it is possible to supply purified gas from the gas pressurizing chamber to each of the housing chambers.

However, in the incubator disclosed in Japanese Laid-Open Patent Application No. 2022-184127, upper and lower housing chambers communicate with each other via an exhaust port provided in the bottom face of the upper housing chamber, so that the gas passed through the upper housing chamber flows into the lower housing chamber via the exhaust port.

Accordingly, when a droplet occurs from a culture container in the upper housing chamber, for example, the droplet flows into the lower housing chamber, possibly causing a cross contamination between the culture containers housed in housing chambers positioned in the up-and-down direction.

In view of the problem described above, the present disclosure provides an incubator capable of preventing cross-contaminations as effectively as possible.

### SUMMARY OF THE INVENTION

An incubator according to the invention of claim 1 includes a plurality of housing chambers that are capable of housing culture containers therein and are arranged in up-and-down and left-and-right directions. The incubator has an opening/closing member provided in front of each of the housing chambers.

The incubator is characterized by including: a gas pressurizing chamber communicating with the housing chambers via a gas supply port provided in a rear face of each of the housing chambers; a gas merging chamber provided in communication with the gas pressurizing chamber; and a gas delivery unit for purifying gas in the gas merging chamber and delivering the purified gas to the gas pressurizing chamber.
an exhaust passage is provided for each of the housing chambers to allow individual communication with the gas merging chamber, so that gas in each of the housing chambers is discharged into the gas merging chamber via the exhaust passage.

### Advantageous Effects of Invention

According to the invention described above, the housing chambers are allowed to individually communicates with the gas merging chamber via the exhaust passage. It is therefore possible to prevent cross-contaminations between the different housing chambers even if a droplet occurs from a culture container in a housing chamber being required.

### BRIEF DESCRIPTION OF THE DRAWINGS

FIG. 1 is a perspective view of an incubator according to an embodiment of the present disclosure;

FIG. 2 is a cross-sectional view of the incubator in lateral-face direction; and

FIG. 3 is a cross-sectional view of the incubator in front-face direction.

### DETAILED DESCRIPTION OF THE INVENTION

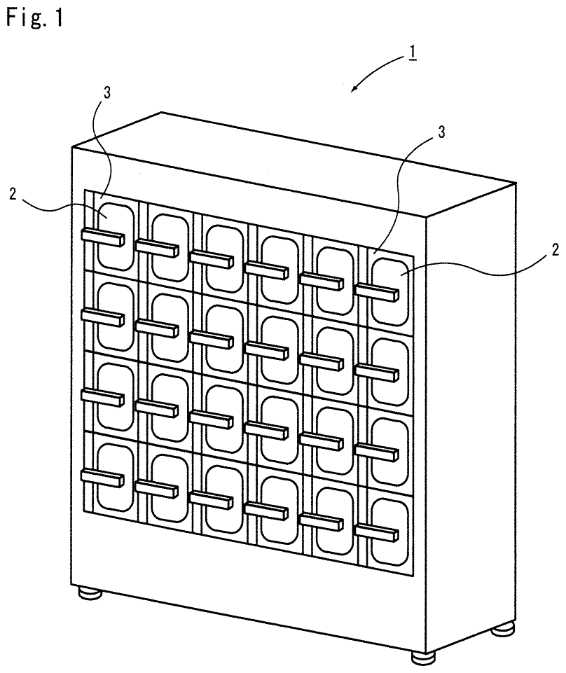

The following will describe an embodiment shown in the drawings. FIG. 1 is a perspective view of an incubator 1 configured to culture cells. The incubator 1 is provided in a culture room of which the interior has been purified.

The incubator 1 includes a plurality of housing chambers 3 that can be opened and closed by opening/closing members 2. In the present embodiment, the incubator 1 includes 24 housing chambers 3 in total that are arranged on four levels in the up-and-down direction and in six columns in transversal directions. In addition, the opening/closing members 2 of the housing chambers 3 are provided on the front face of the incubator 1.

Further, in the culture room, a plurality of AIVs (Autonomous Intelligent Vehicles or autonomous automatic transport robots) (not shown) are also provided in addition to the incubator 1. It is therefore possible to automatically take culture containers 4 in and out of the housing chambers 3 by using robot hands provided for the AIVs.

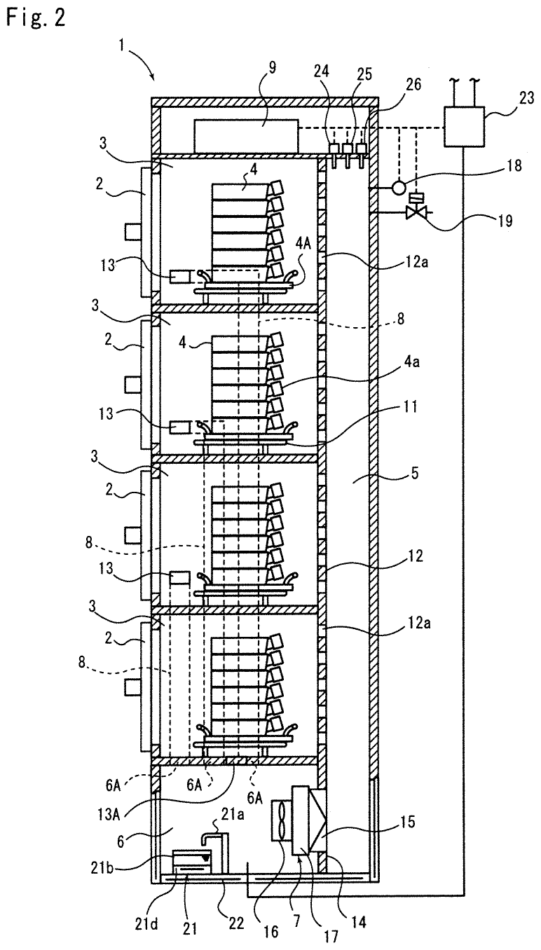

As shown in FIG. 2, each of the culture containers 4 housed in the housing chambers 3 is a tightly-sealable container having a flat shape. It is possible to have a plurality of culture containers 4 housed in each of the housing chambers 3, while being stacked on a tray 4A.

A cap 4a is attached to a tip end of each of the culture containers 4 in the present embodiment. The cap 4a is provided with a filter (not shown) that passes only gas. With this configuration, while preventing the interior of the culture containers 4 from being contaminated, it is possible to cause a gas suitable for the culturing of cells and for flowing through the housing chambers 3 to flow into the culture containers 4.

When the incubator 1 of the present embodiment is used, it is not necessarily requisite to tightly seal the culture containers 4 with caps. It is also possible to use culture containers 4 that are open to the outside, such as petri dishes or other kinds of dishes.

The incubator 1 in the present embodiment includes a gas pressurizing chamber 5 provided at the rear of the housing chambers 3; a gas merging chamber 6 provided below the housing chambers 3; and a gas delivery unit 7 for purifying gas in the gas merging chamber 6 and delivering the purified gas to the gas pressurizing chamber 5. The gas delivery unit 7 is configured to be controlled by a control unit 9.

With this configuration, as a result of the gas delivery unit 7 being operated to deliver the gas from the gas merging chamber 6 to the gas pressurizing chamber 5, the gas is supplied from the gas pressurizing chamber 5 to the housing chambers 3 and is exhausted from the housing chambers 3 to the gas merging chamber 6. In this manner, the gas is circulated while being purified by the gas delivery unit 7.

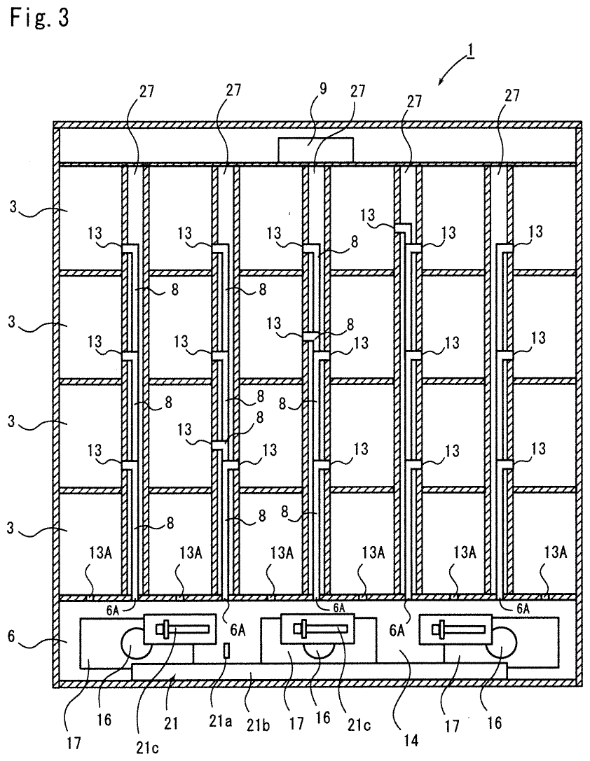

As shown in FIG. 3, each of the housing chambers 3 is surrounded by dividing walls provided at the top and the bottom and on the left and the right. On the front side of each of the housing chambers 3 shown in FIG. 2, the opening/closing member 2 is provided so as to be able to open and close. Each of the opening/closing members 2 is pivotally supported by a hinge (not shown) and is able to automatically open and close via motor drive.

Provided inside each of the housing chambers 3 is a table 11 for supporting the tray 4A on which the culture containers 4 are placed. The tray 4A is provided with a grip part which can be gripped by the robot hands of the AIVs.

With this configuration, at the time of taking the culture containers 4 in and out of any of the housing chambers 3 of the incubator 1, when one of the AIVs moves to the front of a housing chamber 3 being required, the opening/closing member 2 of the housing chamber 3 being required is automatically opened, so that a robot hand grips the tray 4A stacked with a plurality of culture containers 4 and supported by the AIV and places the tray 4A onto the table 11 in the housing chamber 3, or so that a robot hand grips the tray 4A placed on the table 11 and takes out the plurality of stacked culture containers 4 from the housing chamber 3.

The gas pressurizing chamber 5 is provided adjacent to the housing chambers 3 at the rear thereof, and is defined by a single space formed at the rear of all the 24 housing chambers 3 arranged in the up-and-down and the left-and-right directions.

The rear faces of the housing chambers 3 facing the opening/closing members 2 are made of a breathable wall 12 provided with a large number of gas supply ports 12a. While dividing the housing chambers 3 from the gas pressurizing chamber 5, the breathable wall 12 also allows communication therebetween via the gas supply ports 12a.

It is possible to use a punched metal sheet as the breathable wall 12. In that case, the gas supply ports 12a are uniformly arranged in the breathable wall 12 in the up-and-down and the left-and-right directions. The total opening area of the gas supply ports 12a is equal among the housing chambers 3.

By making the total opening area of the gas supply ports 12a equal among the housing chambers 3, it is possible to cause the gas in the gas pressurizing chamber 5 maintained in a pressurized state to flow into the housing chambers 3 at a substantially equal flow rate.

Alternatively, one gas pressurizing chamber 5 may be provided separately for each column of housing chambers 3 arranged in the up-and-down direction (in which case, the gas pressurizing chamber 5 is divided into six columns), or may be provided for each set of three columns of housing chambers 3 (in which case, the gas pressurizing chamber 5 is divided into two columns).

The gas merging chamber 6 is provided adjacent to and immediately underneath the housing chambers 3 positioned on the lowest level. As shown in FIG. 3, the gas merging chamber 6 is formed by a single space extending over the entire width of the six columns of housing chambers 3 arranged in the transversal directions.

Further, the gas merging chamber 6 and the gas pressurizing chamber 5 are divided while a partition wall 14 is interposed therebetween. The partition wall 14 is provided with purification filters 15 serving as the gas delivery unit 7. The gas merging chamber 6 and the gas pressurizing chamber 5 are configured to communicate with each other via the purification filters 15.

In other words, the gas pressurizing chamber 5 has a width extending over the entire width of the six columns of housing chambers 3 arranged in the transversal directions and has a height equal to the height of the gas merging chamber 6 plus the height of the four levels of housing chambers 3 arranged in the up-and-down direction, as shown in FIG. 2.

Further, the bottom face of each of the housing chambers 3 on the first level as the lowest level is provided with an exhaust port 13A, so as to allow direct communication with the gas merging chamber 6 positioned immediately underneath using the exhaust ports 13A as exhaust passages.

In contrast, each of the housing chambers 3 on the second to the fourth levels is provided with an exhaust port 13 formed in one of the left and right lateral faces. The exhaust ports 13 and the gas merging chamber 6 communicate via exhaust pipes 8, each of which defines an exhaust passage.

With this configuration, all the housing chambers 3 individually communicate with the gas merging chamber 6 via the exhaust ports 13A and the exhaust pipes 8.

The gas delivery unit 7 includes: the purification filters 15 provided at the partition wall 14 dividing the gas pressurizing chamber 5 and the gas merging chamber 6; a fan 16 provided in the gas merging chamber 6, and box-like plenums 17 for causing the gas blown by the fan 16 to spread throughout the entirety of the purification filters 15.

As shown in FIG. 3, the partition wall 14 is provided with three plenums 17 arranged in the width direction of the gas merging chamber 6. As a result of operation of the fan 16, the gas in the gas merging chamber 6 fills the plenums 17 and also flows through the purification filters 15, so as to be purified while flowing into the gas pressurizing chamber 5. For the purification filters 15, high performance purification filters such as HEPA or ULPA filters can be used.

Further, the gas pressurizing chamber 5 is provided with a pressure gauge 18 and a pressure reducing valve 19 for discharging the gas from the gas pressurizing chamber 5.

When the gas is supplied by the fan 16 from the gas merging chamber 6 to the gas pressurizing chamber 5, the pressure in the gas pressurizing chamber 5 increases; however, because the control unit 9 opens the pressure reducing valve 19 in accordance with a measurement result obtained by the pressure gauge 18, it is possible to maintain the interior of the gas pressurizing chamber 5 at a prescribed set pressure.

In contrast, when the opening/closing member 2 of one of the housing chambers 3 is opened and the pressure in the housing chamber 3 drops, the pressure in the gas pressurizing chamber 5 also drops. Accordingly, the control unit 9 closes the pressure reducing valve 19 so as to return the reduced pressure in the gas pressurizing chamber 5 to the abovementioned set pressure.

As an adjustment unit for maintaining the internal atmosphere of the housing chambers 3 in an environment suitable for culturing, the incubator 1 of the present embodiment includes: a humidification unit 21 for humidifying the gas to be supplied to the housing chambers 3; a heating unit 22 for heating the interiors of the housing chambers 3, the gas pressurizing chamber 5, and the gas merging chamber 6; and a gas switching unit 23 for supplying one of carbon dioxide and air to the housing chambers 3 by switching between the two.

The humidification unit 21 includes: a water supply nozzle 21a provided in the gas merging chamber 6 for dripping humidification water; an evaporation dish 21b for evaporating the dripped humidification water; sterilization lamps 21c for sterilizing the humidification water in the evaporation dish 21b; and a warmer 21d for warming the evaporation dish 21b and evaporating water to be evaporated.

The water supply nozzle 21a is for dripping sterile water or RO water. The amount of humidification water to be dripped is controlled by the control unit 9. The dripped humidification water is sterilized by the sterilization lamps 21c and vaporized in the evaporation dish 21b. The generated vapor is added to the gas flowing through the gas merging chamber 6, so that the gas adjusted to have humidity suitable for the culturing is supplied to the housing chambers 3.

The heating unit 22 is configured with heater wires embedded in the floor, the ceiling, and wall portions constituting the housing chambers 3, the gas pressurizing chamber 5, and the gas merging chamber 6. Under control of the control unit 9, the heating unit 22 maintains the interiors of the housing chambers 3, the gas pressurizing chamber 5, and the gas merging chamber 6 at a temperature suitable for culturing (approximately 37 °C).

The gas switching unit 23 is configured to be able to switch between purified air and carbon dioxide gas (CO₂) supplied from a supply source (not shown) in a compressed state, by blocking or passing one of the two.

The carbon dioxide prevents oxidization of a culture medium that may be caused by metabolism of cells and adjusts the pH level of the culture medium housed in the culture containers 4 to be in an optimal state. Other than carbon dioxide, it is also possible to use other gas such as nitrogen gas corresponding to the cells to be cultured.

Further, under the control of the control unit 9, by switching between the air and the carbon dioxide and supplying it to the gas merging chamber 6, the gas switching unit 23 is configured to add one of the two to the gas passing through the gas merging chamber 6 and to adjust the gas concentration to be suitable for the culturing before the gas is supplied to the housing chambers 3.

The gas pressurizing chamber 5 is provided with a temperature sensor 24, a humidity sensor 25, and a gas concentration sensor 26, for the purpose of measuring whether or not the gas housed therein is in a state suitable for the culturing of the cells.

These sensors are provided in certain positions in the gas pressurizing chamber 5 away from the gas delivery unit 7. By measuring the state of the gas in the positions distant from the gas delivery unit 7, it is possible to check to see whether the gas is sufficiently agitated and homogenized inside the gas pressurizing chamber 5.

Further, on the basis of measurement results obtained by these sensors, the control unit 9 is configured to control the humidification unit 21, the heating unit 22, and the gas switching unit 23.

The exhaust pipes 8 are provided so as to allow individual communication between the gas merging chamber 6 and the housing chambers 3 on the second to the fourth levels positioned at the levels higher than the housing chambers 3 on the first level being the lowest level. One end of each of the exhaust pipes 8 is connected to the exhaust port 13 provided in one of the left and right lateral faces (the left and right dividing walls) of a corresponding one of the housing chambers 3 positioned on the second to the fourth levels. The other ends thereof are connected to inflow ports 6A opened in the ceiling face of the gas merging chamber 6.

In the present embodiment, each of the exhaust ports 13 is provided in a position closer to the front of a corresponding housing chamber 3 and closer to the bottom of the corresponding housing chamber 3. With this configuration, the gas that has flowed in via the plurality of gas supply ports 12a formed in the rear faces of the housing chambers 3 passes from above to below while passing through the housing chambers 3 from the rear to the front, so as to be exhausted through the exhaust ports 13. However, as long as it is one of the lateral faces (the left and right dividing walls) of the housing chambers 3, the positions of the exhaust ports 13 for connecting the exhaust pipes 8 are not particularly limited.

Each of the exhaust pipes 8 is provided inside an exhaust passage chambers 27 formed between housing chambers 3 that are adjacent in the left-and-right direction, on the four levels arranged in the up-and-down direction.

As mentioned earlier, each of the exhaust ports 13 is formed in one of the left and right lateral faces (the left and right dividing walls) of a corresponding one of the housing chambers 3 on the second to the fourth levels. Thus, one end of each of the exhaust pipes 8 provided in the exhaust passage chambers 27 is positioned so as to be connected to the exhaust port 13 of one of the housing chambers 3 positioned on the left and the right of a corresponding one of the exhaust passage chambers 27 on the second to the fourth levels.

In the present embodiment, the housing chambers 3 on the first to the fourth levels shown at the left end in FIG. 3, which is a cross-sectional view of the incubator 1 in the front-face direction, will be referred to as the first column. The housing chambers 3 on the second to the fourth levels positioned in the first column each have the exhaust port 13 formed in the right lateral face (the right dividing wall) thereof in the drawing as seen from the front. One exhaust pipe 8 is connected to each of the three exhaust ports 13.

Further, without overlapping with one another, the three exhaust pipes 8 being connected are housed in the first exhaust passage chamber 27 formed between the first column and the second column of housing chambers 3 that are adjacent to each other.

In contrast, for the housing chambers 3 on the second to the fourth levels positioned in the second column, exhaust pipes 8 are similarly connected to the exhaust ports 13 formed in the right lateral faces (the right dividing walls), as with the first column. The three exhaust pipes 8 are housed in the second exhaust passage chamber 27 formed between the second column and the third column of housing chambers 3 that are adjacent to each other.

However, among the housing chambers 3 on the first to the fourth levels in the third column, the housing chamber 3 on the second level has an exhaust port 13 formed in the left lateral face (the left dividing wall) and has an exhaust pipe 8 connected thereto. Thus, the second exhaust passage chamber 27 has housed therein four exhaust pipes 8 in total.

Furthermore, among the housing chambers 3 in the third column, each of the housing chambers 3 on the third and the fourth levels has an exhaust port 13 formed in the right lateral face (the right dividing wall) thereof. In contrast, among the housing chambers 3 in the fourth column positioned adjacent thereto, each of the housing chambers 3 on the second and the third levels has an exhaust port 13 formed in the left lateral face (the left dividing wall) thereof, whereas the housing chamber 3 on the fourth level has an exhaust port 13 formed in the right lateral face thereof.

As a result, the third exhaust passage chamber 27 formed between the third column and the fourth column of housing chambers 3 that are adjacent to each other, has housed therein two exhaust pipes 8 connected to the exhaust ports 13 of the housing chambers 3 on the third and the fourth levels in the third column positioned on the left; and two exhaust pipes 8 connected to the exhaust ports 13 of the housing chambers 3 on the second and the third levels in the fourth column positioned on the right. Thus, the four exhaust pipes 8 in total are housed.

In other words, some exhaust passage chambers 27 each house therein three exhaust pipes 8 connected to the exhaust ports 13 of the housing chambers 3 in one column positioned on the left or on the right. Other exhaust passage chambers 27 each house therein four exhaust pipes 8 in total that are connected to the exhaust ports 13 of the housing chambers 3 in the two columns positioned on the left and the right. In addition, the exhaust pipes 8 housed in the exhaust passage chambers 27 are provided separately from each other on the inside.

More specifically, because each of the exhaust pipes 8 is formed in a position closer to the front of the corresponding housing chamber 3, as indicated with the dotted line in FIG. 2, the exhaust pipes 8 connected to the housing chambers 3 on the second level are provided so as to simply extend downward, whereas the exhaust pipes 8 connected to the housing chambers 3 on the third level are provided such that they are directed rearward for a prescribed distance from the exhaust ports 13 in order to avoid the exhaust pipes 8 connected to the housing chambers 3 on the second level and are subsequently directed downward.

Similarly, the exhaust pipes 8 connected to the housing chambers 3 on the fourth level are provided such that they are directed rearward for a prescribed distance from the exhaust ports 13 in order to avoid the exhaust pipes 8 connected to the housing chambers 3 on the third level and are subsequently directed downward.

Further, the housing chamber 3 on the fourth level among the fourth column of housing chambers 3 has an exhaust port 13 formed in the right lateral face (the right dividing wall) and has an exhaust pipe 8 connected thereto. As for the housing chambers 3 in the fifth column, the housing chambers 3 on the second to the fourth levels each have an exhaust port 13 formed in the left lateral face (the left dividing wall) and have an exhaust pipe 8 connected thereto. Thus, the four exhaust pipes 8 in total are housed in the fourth exhaust passage chamber 27 formed between the fourth column and the fifth column of housing chambers 3.

Further, the fifth exhaust passage chamber 27 formed between the fifth column and the sixth column of housing chambers 3 has housed therein, with respect to the housing chambers 3 on the second to the fourth levels in the sixth column, three exhaust pipes 8 connected to the exhaust ports 13 formed in the left lateral faces (the left dividing walls).

The other end portions of the exhaust pipes 8 are connected to the inflow ports 6A opened in the top face of the gas merging chamber 6 so that the gas from the housing chambers 3 flows in from above the gas merging chamber 6, including the exhaust ports 13 provided between the housing chambers 3 on the lowest level and the gas merging chamber 6.

Furthermore, it is possible to configure the incubator 1 to be compact by ensuring that all the exhaust pipes 8 are housed in the exhaust passage chambers 27 each provided between housing chambers 3 adjacent in the left-and-right direction and are positioned so as not to overlap with one another in the exhaust passage chambers 27. In addition, because the exhaust pipes 8 are not positioned outside the incubator 1, the outer peripheral surface of the incubator 1 is flat and orderly.

The following will describe a method for using the incubator 1 having the above configuration. The incubator 1 is provided in an unmanned culture room maintained in a purified environment. One of the AIVs (not shown) provided in the culture room transports a plurality of culture containers 4 having the cells housed therein while being stacked on the tray 4A to the front of the incubator 1.

When the AIV stops at a position based on a command, the opening/closing member 2 of one of the housing chambers 3 being required is opened. The AIV grips the tray 4A with a robot hand and places the culture containers 4 onto the table 11 in the housing chamber 3. After that, the opening/closing member 2 is closed.

On the inside (the housing chambers 3, the gas pressurizing chamber 5, and the gas merging chamber 6) of the incubator 1, the gas adjusted to be in a state suitable for the culturing of the cells is circulated while being purified. In this manner, the cells seeded in the culture containers 4 are cultured over a prescribed period of time.

The gas in the gas pressurizing chamber 5 has been purified by the purification filters 15 and is circulated while being adjusted into a state suitable for the culturing of the cells by the humidification unit 21, the heating unit 22, and the gas switching unit 23 serving the abovementioned adjustment unit.

The state of the gas filling the gas pressurizing chamber 5 is measured by the temperature sensor 24, the humidity sensor 25, and the gas concentration sensor 26 provided in the gas pressurizing chamber 5. The control unit 9 is configured to adjust the state of the gas by controlling the humidification unit 21, the heating unit 22, and the gas switching unit 23 on the basis of the measurement results.

Further, the interior of the gas pressurizing chamber 5 is kept in a pressurized state, as a result of the fan 16 of the gas delivery unit 7 adjusting the amount of the gas supplied from the gas merging chamber 6, with respect to the total opening area of the plurality of gas supply ports 12a formed in the breathable wall 12. By controlling the pressure reducing valve 19 while measuring the pressure with the pressure gauge 18, the control unit 9 is configured to maintain the gas pressurizing chamber 5 in the prescribed pressurized state.

With this configuration, even when the opening/closing member 2 of the housing chamber 3 being required is opened and closed so as to house or take out the culture containers 4, and the pressure drops due to the housing chamber 3 having been opened to the outside, it is possible to maintain the pressure inside the gas pressurizing chamber 5 within a prescribed range.

There is a differential pressure between the gas pressurizing chamber 5 and the housing chambers 3 due to the pressurized state set for the gas pressurizing chamber 5. Because of the differential pressure, the gas in the gas pressurizing chamber 5 flows into all the housing chambers 3 via the gas supply ports 12a formed in the breathable wall 12.

In the present embodiment, the gas pressurizing chamber 5 is provided so as to encompass all the housing chambers 3. It is therefore possible to supply the homogenized gas to all the housing chambers 3. It is also possible to substantially equalize the flow rate of the gas to be supplied.

In particular, in the present embodiment, the housing chambers 3 are allowed to individually communicate with the gas merging chamber 6. Because the gas having flowed into each of the housing chambers 3 is individually discharged into the gas merging chamber 6, the differential pressure between the housing chambers 3 and the gas merging chamber 6 is constant.

In contrast, in the configuration disclosed in Japanese Laid-Open Patent Application No. 2022-184127, because the housing chambers 3 aligned in the up-and-down direction communicate with one another through the exhaust ports 13, it is necessary to set the pressure in the housing chamber 3 positioned on the highest level to be higher than the pressure in the housing chambers 3 positioned on the lower levels in order to discharge the gas in the housing chamber 3 on the highest level. Thus, it is necessary to vary the diameters of the gas supply ports 12a in the breathable wall 12 or the like.

The gas having flowed into the housing chambers 3 from the gas pressurizing chamber 5 in this manner passes through the housing chambers 3 from the rear toward the front, while passing the surroundings of the culture containers 4 housed in the housing chambers 3. Accordingly, the internal spaces of the housing chambers 3 are maintained in a fresh state at all times by the purified gas flowing in through the breathable wall 12.

Among the housing chambers 3, the housing chambers 3 on the first level positioned on the lowest level directly communicate with the gas merging chamber 6 via the exhaust ports 13A serving as exhaust passages. The gas having passed through the housing chambers 3 on the lowest level is directly discharged into the gas merging chamber 6 via the exhaust ports 13A.

In contrast, the gas having flowed into the housing chambers 3 positioned on the second to the fourth levels as upper levels is discharged through the exhaust ports 13 provided in the lateral faces (the dividing walls on the left and the right) of the housing chambers 3, so as to be individually discharged into the gas merging chamber 6 via the exhaust pipes 8 connected to the housing chambers 3 and serving as the exhaust passages.

In this manner, in the present embodiment, because each of the housing chambers 3 individually communicates with the gas merging chamber 6 via the exhaust passage, the occurrence of contaminations with the other housing chambers 3 is prevented.

As for the gas merging chamber 6, because the fan 16 of the gas delivery unit 7 blows gas from the gas merging chamber 6 to the gas pressurizing chamber 5, the gas merging chamber 6 is in a state of having a relatively low pressure. Thus, suction force is generated in the exhaust pipes 8 through the exhaust ports 13A and the inflow ports 6A of the housing chambers 3, and a flow of gas is formed inside the housing chambers 3.

The gas having flowed into the gas merging chamber 6 is adjusted by the adjustment unit so as to be in a state suitable for the culturing of the cells and is then delivered to the gas pressurizing chamber 5.

In accordance with the measurement results obtained by the temperature sensor 24, the humidity sensor 25, and the gas concentration sensor 26 provided in the gas pressurizing chamber 5, the control unit 9 controls the humidification unit 21, the heating unit 22, and the gas switching unit 23 so as to adjust the circulated gas to have required levels of humidity, temperature, and carbon dioxide concentration.

The gas that has been adjusted in this manner is delivered from the gas merging chamber 6 to the gas pressurizing chamber 5 by the fan 16 of the gas delivery unit 7. At that time, the gas is circulated while being purified by the purification filters 15 described above. As a result, in comparison to incubators of a type that is configured to constantly supply fresh gas and to exhaust gas, it is possible to significantly reduce the amount of gas used, which is more economical.

Further, the housing chambers 3 positioned immediately above the gas merging chamber 6 each have, in the bottom face thereof, the exhaust port 13A opened so as to directly communicate with the gas merging chamber 6. The higher housing chambers 3 above them are connected to the gas merging chamber 6 via the exhaust pipes 8. In this manner, each of all the housing chambers 3 individually communicates with the gas merging chamber 6 through the exhaust passages.

In this regard, all the exhaust pipes 8 are housed in the exhaust passage chambers 27 each provided between housing chambers 3 that are adjacent in the left-and-right direction, while the exhaust pipes 8 are positioned so as not to overlap each other in the exhaust passage chambers 27. Thus, no pipe is provided on the outside of the incubator 1, allowing the incubator 1 to be compact and to have an orderly external appearance.

Further, another configuration is also possible in which the housing chambers 3 on the first level as the lowest level are provided at an interval from the gas merging chamber 6; each of the housing chambers 3 on the first level has an exhaust port provided in the bottom face thereof; inflow ports are provided, in correspondence therewith, in the ceiling face of the gas merging chamber 6; and exhaust passages are formed by connecting the exhaust ports to the inflow ports with the exhaust pipes 8.

### REFERENCE SIGNS list

1: incubator
2: opening/closing member
3: housing chamber
4: culture container
5: gas pressurizing chamber
6: gas merging chamber
7: gas delivery unit
8: exhaust pipe (exhaust passage)
12: breathable wall
12a: gas supply port
13: exhaust port
13A: exhaust port (exhaust passage)
27: exhaust passage chamber

1. An incubator including a plurality of housing chambers that are capable of housing culture containers therein and are arranged in up-and-down and left-and-right directions, an opening/closing member provided in front of each of the housing chambers, the incubator comprising:
a gas pressurizing chamber communicating with the housing chambers via at least one gas supply port provided in a rear face of each of the housing chambers;
a gas merging chamber provided in communication with the gas pressurizing chamber; and
a gas delivery unit for purifying gas in the gas merging chamber and delivering the purified gas to the gas pressurizing chamber, wherein
an exhaust passage is provided for each of the housing chambers to allow individual communication with the gas merging chamber, so that gas in each of the housing chambers is discharged into the gas merging chamber via the exhaust passage.

2. The incubator according to claim 1, wherein
the exhaust passage is connected to exhaust ports provided in lateral faces of the housing chambers.

3. The incubator according to claim 2, wherein
an exhaust passage chamber housing the exhaust passage therein is provided between housing chambers that are adjacent in left-and-right direction.

4. The incubator according to claim 2, wherein
the gas merging chamber is provided underneath the housing chambers on a lowest level, and
the housing chambers on the lowest level are allowed to individually communicate with the gas merging chamber via exhaust ports provided in bottom faces of the housing chambers on the lowest level.
